(19) United States
(12) Patent Application Publication
(10) Pub. No.: US 2023/0070984 A1
(43) Pub. Date: Mar. 9, 2023

(54) OXYGEN/CARBON DIOXIDE COMPOSITIONS FOR MEDICAL USES

(71) Applicants: AVIATION WORKS LIMITED, London (GB); UNIVERSITY OF ZURICH, Zurich (CH)

(72) Inventors: Marc STUDER, London (GB); Simisola OKE, London (GB); Rob BOYLE, London (GB); Beatrice Beck SCHIMMER, Adliswil (CH); Martin SCHLÄPFER, Zumikon (CH)

(73) Assignees: AVIATION WORKS LIMITED, London (GB); UNIVERSITY OF ZURICH, Zurich (CH)

(21) Appl. No.: 17/800,350

(22) PCT Filed: Feb. 18, 2021
(86) PCT No.: PCT/EP2021/053989

§ 371 Date: Aug. 17, 2022

(30) Foreign Application Priority Data
Feb. 18, 2020 (EP) 20157869.7

(51) Int. Cl. A61K 33/00; A61P 41/00

(52) U.S. Cl. CPC A61K 33/00; A61P 41/00

(57) ABSTRACT

The present invention relates to compositions comprising gases, particularly oxygen (O2) in an amount of from about 91% to about 99% by volume, and carbon dioxide (CO2) in an amount of from about 1% to about 9% by volume, based upon the total volume of the composition, and their use in mitigating the negative effects of surgery and/or anaesthesia on a patient.

### FIELD OF THE INVENTION

The present invention relates to compositions comprising gases, particularly oxygen (O₂) and carbon dioxide (CO₂), and their use in mitigating the negative effects of surgery and/or anaesthesia on a patient.

### BACKGROUND

Before the induction of general anaesthesia, patients may be pre-oxygenated with 100% oxygen to maximise their oxygen reserve and to allow longer period of apnoea to be tolerated without severe consequences (increasing apnoea tolerance). This lowers the risk of hypoxia and brain damage, should ventilation fail.

Carbon dioxide may be added to a hypoxic gas mixture to accelerate respiratory and heart rate, increasing cardiac output, while decreasing ventilation-perfusion (V/Q) mismatch and serum lactate levels. The effect of CO₂ in humans has mostly been studied under hypobaric hypoxic conditions in relation to altitude sickness, or in aerospace medicine studies to maintain cognitive function in individuals performing complex tasks in hypoxic environments.

Compositions comprising oxygen and carbon dioxide have been used to stimulate respiration after a period of apnoea and in the management of chronic respiratory obstruction after the obstruction has been removed.

One of the problems associated with depriving the body of oxygen, and in particular depriving the brain of oxygen (such as in hypoxia), is that the body and brain can suffer irreparable damage. As the brain is poor at storing oxygen, this may occur within a short time period. There is therefore a need to provide improved care to protect patients' bodies, and in particular their brains, from imminent damage during times when oxygen deprivation to the body, and in particular the brain, may be reduced. This can occur due to complications during surgery or complications during anaesthesia.

### BRIEF DESCRIPTION OF THE DRAWINGS

FIG. 1 is a graphical display of the patient's study intervention of Example 1.

FIG. 2 shows the time until the peripheral oxygen saturation (SpO₂) reached 80% (FIG. 2A), cerebral tissue oxygenation index (TOI) (FIG. 2B), arterial oxygen pressure (PaO₂) (FIG. 2C), and arterial carbon dioxide pressure (PaCO2) (FIG. 2D) at the end of the apnoea phase of all patients in the cross over study in Example 1. The time until SpO₂ reached 80% was similar after the two treatments, while TOI (p=0.02), PaO₂ (p=0.02), and PaCO₂ (p<0.001) increased under 5% CO₂/95% O₂ administration.

FIG. 3 shows that the application of 5% CO₂/95% O₂ administration leads to a carry over-effect. In the sequence control-5% CO₂/95% O₂ (A, n=17), brain tissue oxygenation (TOI) values did not change from baseline (T0) to T1 (prior to the first apnoea) but are higher at T3 (after 5% CO₂/95% O₂ administration), prior to the second apnoea. Whereas in the sequence 5% CO₂/95% O₂-control (B, n=13), TOI values increase from baseline (T0) to T1 (after 5% CO₂/95% O₂ administration) and remain at the level of T1, even after washout of 5% CO₂/95% O₂ and prior to the second apnoea phase (T3).

### DISCLOSURE OF THE INVENTION

Surprisingly, it has been found that administering a composition comprising oxygen and carbon dioxide to patients (subjects) undergoing certain surgeries, or subjects at risk of suffering apnoea during anaesthesia, provides certain benefits, such as reduced severity, or removal of, certain complications associated with said surgery and/or apnoea.

Consequently, in a first aspect of the invention there is provided a composition comprising oxygen (O₂) and carbon dioxide (CO₂), wherein
(i) O₂ is present in an amount of from about 91% to about 99% by volume; and
   (ii) CO₂ is present in an amount of from about 1% to about 9% by volume,
      based upon the total volume of the composition.

It is preferred that the O₂ is present in an amount of from about 93% to about 97% by volume; and the CO₂ is present in an amount of from about 3% to about 7% by volume. It is more preferred that the O₂ is present in an amount of from about 94% to about 96% by volume; and the CO₂ is present in an amount of from about 4% to about 6% by volume.

It is essential that the composition comprises O₂ and CO₂, however, additional components may be included in the composition, such as a carrier gas. A carrier gas is a gas, or mixture of gases, that has no, or minimal, therapeutic effect on the subject. It may be present in the composition in an amount of from greater than 0% to about 8%, preferably from greater than 0% to about 4%, more preferably from greater than 0% to about 2%, by volume based upon the total volume of the composition. It is preferable that carrier gas comprises nitrogen (N₂), helium (He), xenon (Xe), argon (Ar), krypton (Kr) or a mixture thereof.

As will be understood, O₂ and CO₂ (and the carrier gas) will be in their gaseous state at atmospheric pressure and a temperature of 25° C. It is therefore preferable that the composition of the first aspect of the invention is provided as a liquefied gas in a suitable container, such as a gas cylinder.

A particular feature of the first aspect of the invention is that the composition consists of O₂ and CO₂. As used herein, the term “consists of” means that O₂ and CO₂ are the only components forming the composition, however, it does not preclude other components from being present in the composition as impurities. Such impurities may be present in amount of less than about 1%, preferably less than about 0.5%, more preferably less than about 0.2%, even more preferably less than about 0.1% and most preferably less than about 0.05%, by volume based upon the volume of the composition.

It will be understood that the composition of the first aspect of the invention may be mixed with other gases, such as anaesthetics (particularly volatilised anaesthetics) and/or other medicaments during or prior to its use.

In the most preferred embodiment of the first aspect of the invention, the composition does not consist of 95% by volume of O₂ and 5% by volume of CO₂ based upon the total volume of the composition.

As the composition is intended to be used as a medicament, it is preferable that it is provided as a medical grade composition.

Those skilled in the art will understand how to produce a composition of the invention. In particular, the composition may be formed by providing either oxygen or carbon dioxide in a suitable container, such as a gas cylinder, and introducing the remaining components until the desired mixture is obtained. The amount of gas added to the container may be measured by volume (for instance using a flow meter) or by weight.

The compositions of the first aspect of the invention may be referred to as a composition of the invention.

In a second aspect of the invention there is provided the composition of the invention for use as a medicament.

It has been surprisingly found that the composition of the first aspect of the invention has particular advantages when administered to subjects undergoing certain surgeries and/or anaesthesia. These surgeries include those during which it is desirable that the subject has a low blood pressure, surgeries that are expected to result in high blood loss, and/or surgeries that requires relaxed blood vessels. The composition of the invention has been found to have particular utility in subjects undergoing anaesthesia, especially those at risk of suffering apnoea during anaesthesia.

Therefore, in a third aspect of the invention, there is provided a composition of the invention for use
(i) in the treatment or prevention of at least one complication associated with surgery on a subject; and/or
(ii) in, or prior to, surgery on a subject,
   wherein it is desirable that the subject has low blood pressure during the surgery; the surgery is expected to result in high blood loss; and/or the surgery requires relaxed blood vessels.

Additionally, in the third aspect of the invention, there is provided a composition of the invention for use in
(i) the treatment or prevention of at least one complication associated with apnoea in a subject; and/or
(ii) increasing apnoea tolerance in a subject,
   wherein the apnoea is during, or expected during, anaesthesia.

Without wishing to be bound by theory, in systemic capillaries, an increase in carbon dioxide partial pressure (pCO₂) and temperature, as well as a decrease in pH, will lower haemoglobin (Hb) affinity for oxygen which facilitates its unloading into tissue. Further, high CO₂ promotes vasodilatation of arterioles, which may further increase oxygen delivery to tissues. It is believed that this not only happens systemically, but is an essential component of cerebral autoregulation in case of hypercarbia and/or acidemia, both of which increase cerebral blood flow (CBF). Hypoxia likewise may stimulate vasodilation, and concurrent hypoxia and hypercarbia may result in a greater increase in CBF compared to hypoxia alone. In view of this, inhalation of the composition of the invention by a subject may lead to improve cerebral oxygenation. Therefore, it is believed that providing carbon dioxide before (preoperatively) or during (perioperatively) surgery and/or anaesthesia may help protect a subject from complications caused by depriving the body, in particular the brain, of adequate oxygen supply. The composition of the invention may therefore be administered to a subject to reduce perioperative risk.

Based upon the above, in a feature of the third aspect of the invention, the composition of the invention is administered to the subject before and/or during surgery.

A greater reduction in perioperative risk may be achieved by administering the composition of the invention to the subject prior to the start of surgery (i.e. before surgery), and more preferable prior to the start of surgery and prior to the administration of anaesthetic (i.e. before both surgery and anaesthesia). In this case, the composition is used preventatively.

As used herein, the terms “treatment,” “treating,” “treat” and the like, refer to obtaining a desired pharmacologic and/or physiologic effect. In the case of the treatment of a complication arising from surgery or apnoea, the effect can be prophylactic in terms of completely or partially preventing said complication or a symptom thereof and/or can be therapeutic in terms of a partial or complete cure for complication and/or an adverse effect attributable to the surgery or apnoea.

As used herein, the term “prevention” refers to the intention to prevent negative effects of surgery or apnoea, such complications arising therefrom. “Prevention” also includes the intention to reduce the effect of a complication arising from surgery or apnoea on the subject. In both cases it includes “prophylactic treatment”, for instance of certain complications.

The term “at least one” may also be described as “one or more”, i.e. 1, 2, 3, 4, 5, 6, 7, 8, 9, 10 or more.

The term “complication” refers to a secondary disease or condition that develops in the course of a primary event, such as apnoea brought on by anaesthesia or by the act of surgery. Complications include, but are not limited to, oxygen deficiency, such as limited blood flow or oxygen to the heart or brain and cerebral hypoxia; diabetes; transient ischemic attack (TIA); stroke; and heart attack. Certain complications may lead to death of the subject.

Those skilled in the art will understand the term “surgery”. It includes the treatment of injuries, diseases or conditions suffered by a subject, typically by incision or manipulation, especially with instruments.

The phrase “in, or prior to, surgery” means during the surgery or immediately prior to the surgery, for instance when the subject is being prepared for surgery. “Prior to surgery” means within 6, 5, 4, 3, 2, 1, hour before the start of surgery, preferably within 30 minutes before the start of surgery.

The term “subject” means patient. The subject is preferably a mammal, and more preferably a human.

The term “high blood loss” includes that required to drop blood pressure is Class 3 and 4 which nominally is more than 30% blood loss or more than 1500 mL.

The term “apnoea” is known in the art and includes the temporary cessation of breathing. It is preferable that apnoea refers to that in a subject under anaesthesia.

The term “anaesthesia” as used herein is a state of controlled, temporary loss of sensation or awareness that is induced for medical purposes. It may include analgesia (relief from or prevention of pain), paralysis (muscle relaxation), amnesia (loss of memory), or unconsciousness. It is preferable that the anaesthesia is general anaesthesia which is a state of controlled unconsciousness.

Suitable anaesthetics useful in the invention include lidocaine (for instance, for sedation and systemic analgesia for intubation), fentanyl (for instance, for systemic analgesia for intubation), propofol (for instance, for sedation for intubation), and mixtures of oxygen and inhalational anaesthetics, and combinations thereof. Inhalational anaesthetics include desflurane, isoflurane, nitrous oxide, sevoflurane, xenon, chloroethane (ethyl chloride), chloroform, cryofluorane, cyclopropane, diethyl ether, divinyl ether, enflurane, ethylene, Fluroxene, Halothane, methoxyflurane, methoxypropane, trichloroethylene, divinyl ether, and mixtures thereof, preferably desflurane, isoflurane, nitrous oxide, sevoflurane, xenon and mixtures thereof.

Maintaining blood pressure is critical during certain surgeries. This may be for a number of reasons, which include the increased likelihood of increasing or decreasing blood pressure due to surgical techniques or drugs provided to the subject. It is beneficial in certain surgeries that the subject has low or lower blood pressure. These include, but are not limited to, ophthalmic surgery, neurosurgery, vascular surgery and otorhinolaryngology surgery on the middle ear, amongst others.

Of particular use in the present invention is the composition for use in vascular surgery that requires relaxed blood vessels. Such surgery may involve arterial catheterisation, preferably wherein the surgery involves cardiac angiogram, cardiac stent placement, rotablation, and/or intra-arterial balloon tamponading.

It will be understood that surgeries that require a low blood pressure include those in which it is preferable that the blood pressure of the subject is less than 90 mmHg systolic, such as less than 80 mmHg, or even lower.

A problem with conducting surgery on subjects with low blood pressure, or surgeries that result in high blood loss, is ensuring that the brain receives enough oxygen. The present invention may alleviate this problem.

Further, certain surgeries carry a greater risk of high blood loss. These include orthopaedic surgery, vascular surgery, obstetric surgeries and any surgery requiring an open technique.

Subjects who are obese and/or have low functional residual lung capacity (FRC) may be particularly at risk of reduce supply of oxygen to their body, especially the brain. This may be, for instance, due to their increased vulnerability from hypoxia in the case of apnoea (particularly prolonged apnoea). Further, obese patients may have a low FRC, for instance in the supine position, due to the added tissue weight opposing the outward recoil of the chest wall. The present invention may present a particular benefit for these subjects. These subjects may benefit most from administration of the composition of the invention prior to the start of surgery (i.e. before surgery), and more preferable prior to the start of surgery and prior to the administration of anaesthetic (i.e. before both surgery and anaesthesia).

As used herein, the term “obese” includes subjects who have a Body Mass Index (BMI) of greater than 35 kg/m³. As is known, BMI is a value derived from the mass (weight) and height of a person. BMI is defined as the body mass divided by the square of the body height, and is universally expressed in units of kg/m², resulting from mass in kilograms and height in metres. The present invention may be particularly beneficial to subjects with a BMI of greater than 40 kg/m³, such as those with a BMI of greater than 45 kg/m³

The term “functional residual lung capacity” (FRC) is the volume of air present in the lungs at the end of passive expiration. At FRC, the opposing elastic recoil forces of the lungs and chest wall are in equilibrium and there is no exertion by the diaphragm or other respiratory muscles. FRC is the sum of expiratory reserve volume (ERV) and residual volume (RV) and measures approximately 2100 mL in a 70 kg, average-sized male (or approximately 30 mL/kg). A subject with low FRC is classified as having an FRC of less than 30 mL/kg when measured in the supine position. FRC may be measured by spirometry.

In view of the above, the present invention may be particular useful in bariatric surgery. The term “bariatric surgery” includes, but is not limited to, surgery relating to the placement of a gastric band around the stomach, so that the subject does not need to eat as much to feel full; gastric bypass during which the top part of the stomach is joined to the small intestine, so that the subject feels fuller sooner and does not absorb as many calories from food; and sleeve gastrectomy during which some of the stomach is removed, so that the subject cannot eat as much as they could and feels full sooner.

A particular feature of the third aspect of the invention is that the composition is administered to the subject under normobaric conditions. The term “normobaric conditions” means at a constant atmospheric pressure.

As mentioned, the composition of the invention may be administered when the subject is under anaesthesia (preferably general anaesthesia), or before anaesthesia (preferably general anaesthesia) is administered to the subject. It may be particularly advantageous to administer the composition of the invention before the anaesthesia is administered to the subject. This may, for instance, increase the subject's apnoea tolerance while under anaesthesia, and in particular while under general anaesthesia.

The composition should be administered to the subject as a gas and by inhalation. This would typically be done within a surgical environment. Apparatus for administering gases to a subject within a surgical environment are well known to the skilled person, and in particular to anaesthetists.

In a particular feature of third aspect of the invention, the composition comprises O₂ and CO₂, wherein
(i) O₂ is present in an amount of from about 91% to about 99% by volume, preferably from about 93% to about 97% by volume; and
(ii) CO₂ is present in an amount of from about 1% to about 9% by volume, preferably from about 3% to about 7% by volume,
   based upon the total volume of the composition,
   and the composition is for use in
(a) the treatment or prevention of at least one complication associated with surgery on a subject; and/or
(b) surgery on a subject,
   wherein it is desirable that the subject has low blood pressure during the surgery; the surgery is expected to result in high blood loss; and/or the surgery requires relaxed blood vessels,
   and wherein the composition is administered to the subject before surgery. When it is desirable that the subject has low blood pressure during the surgery, it is preferably that the blood pressure is less than 90 mmHg systolic. In this particular feature, the subject may be obese, preferably having a BMI of greater than 35 kg/m³; and/or have low FRC, preferably an FRC of less than 30 mL/kg when measured in the supine position.

In another particular feature of third aspect of the invention, the composition comprises O₂ and CO₂, wherein
(i) O₂ is present in an amount of from about 91% to about 99% by volume, preferably from about 93% to about 97% by volume; and
(ii) CO₂ is present in an amount of from about 1% to about 9% by volume, preferably from about 3% to about 7% by volume,
   based upon the total volume of the composition,
   and the composition is for use in
(i) the prevention of at least one complication associated with apnoea in a subject; and/or
(ii) increasing apnoea tolerance in a subject,
   wherein the apnoea is during, or expected during, anaesthesia,
   wherein the composition is administered to the subject before anaesthesia. In this particular feature, the subject may be obese, preferably having a BMI of greater than 35 kg/m³; and/or have low FRC, preferably an FRC of less than 30 mL/kg when measured in the supine position.

In view of the advantages offered by the present invention, the composition are particularly useful
1) in, or prior to, bariatric surgery;
2) in the treatment or prevention of a complication selected from the group consisting of oxygen deficiency, such as limited blood flow or oxygen to the heart or brain and cerebral hypoxia; high blood pressure; diabetes; transient ischemic attack (TIA); stroke; heart attack; and death; and/or
3) when administered to a subject before and/or during surgery, preferably before surgery, more preferably before both surgery and anaesthesia.

Most preferably, the invention relates to
(i) prevention of at least one complication associated with apnoea; and/or
(ii) increasing apnoea tolerance in a subject,
   wherein the apnoea is during, or expected during, anaesthesia, and in particular bariatric surgery under general anaesthesia during which apnoea is expected to, or does, occur.

For the avoidance of doubt, any composition of the invention may be used in the third aspect of the invention. Whilst a composition consisting of 95% by volume of O₂ and 5% by volume of CO₂ may be excluded from the first aspect of the invention, that composition is particularly preferred in the medical uses mentioned. The composition consisting of 95% by volume of O₂ and 5% by volume of CO₂ may be referred to as “oxycarbon”.

In a fourth aspect of the invention, there is provided a composition of the invention for use in the manufacture of a medicament for the uses as defined in relation to the third aspect of the invention, each combination of which is herein disclosed.

In a fifth aspect of the invention, there is provided a method for treating or preventing the complications, performing surgery on a subject, or increasing apnoea tolerance in a subject, in line with the uses as defined in relation to the third aspect of the invention each combination of which is herein disclosed.

In view of the fourth and fifth aspects of the invention, the following are non-limiting numbered embodiments of the present invention.

### Fourth Aspect Embodiments

### Embodiment 1

Use of a composition of the first aspect of the invention in the manufacture of a medicament for use
(i) in the treatment or prevention of at least one complication associated with surgery on a subject; and/or
(ii) in, or prior to, surgery on a subject,
   wherein it is desirable that the subject has low blood pressure during the surgery; the surgery is expected to result in high blood loss; and/or the surgery requires relaxed blood vessels, preferably wherein the blood pressure is less than 90 mmHg systolic.

### Embodiment 2

The use of a composition of the first aspect of the invention in the manufacture of a medicament for use in
(i) the treatment or prevention of at least one complication associated with apnoea in a subject; and/or
(ii) increasing apnoea tolerance in a subject,
   wherein the apnoea is during, or expected during, anaesthesia.

### Embodiment 3

The use of embodiment 1, wherein the surgery is selected from the group consisting of ophthalmic surgery, neurosurgery, vascular surgery and otorhinolaryngology surgery on the middle ear, amongst others.

### Embodiment 4

The use of embodiment 3, wherein the use is in vascular surgery that requires relaxed blood vessels, and the surgery involves arterial catheterisation, preferably wherein the surgery involves cardiac angiogram, cardiac stent placement, rotablation, and/or intra-arterial balloon tamponading.

### Embodiment 5

The use of embodiment 1 or embodiment 2, wherein the use is in, or prior to, surgery, and the surgery is bariatric surgery.

### Embodiment 6

The use of any one of embodiments 1 to 5, wherein the at least one complication is selected from the group consisting of oxygen deficiency, such as limited blood flow or oxygen to the heart or brain and cerebral hypoxia; high blood pressure; diabetes; transient ischemic attack (TIA); stroke; heart attack; and death.

### Embodiment 7

The use of any one of embodiments 1 to 6, wherein the composition is administered to the subject before and/or during surgery, preferably before surgery, more preferably before both surgery and anaesthesia.

### Embodiment 8

The use of any one of embodiments 1 to 7, wherein the subject
(i) is obese, preferably having a Body Mass Index (BMI) of greater than 35 kg/m³; and/or
(ii) has low functional residual lung capacity (FRC), preferably an FRC of less than 30 mL/kg when measured in the supine position.

### Embodiment 9

The use of any one of embodiments 1 to 8, wherein, in use, the composition is administered to the subject under normobaric conditions.

### Embodiment 10

The use of any one of embodiments 1 to 9, wherein the use is when the subject is under general anaesthesia, or before general anaesthesia is administered, preferably before general anaesthesia is administered.

### Embodiment 11

The use of any one of embodiments 1 to 10, wherein the composition is administered to the subject as a gas by inhalation.

### Embodiment 12

The use of any one of embodiments 1 to 11, wherein the composition consists of 95% by volume of O₂ and 5% by volume of CO₂.

### Fifth Aspect Embodiments

### Embodiment 13

A method for
(i) the treatment or prevention of at least one complication associated with surgery on a subject; and/or
(ii) performing surgery on a subject,
   comprising administering to the subject a composition of the first aspect of the invention in, or prior to, the surgery, wherein it is desirable that the subject has low blood pressure during the surgery; the surgery is expected to result in high blood loss; and/or the surgery requires relaxed blood vessels, preferably wherein the blood pressure is less than 90 mmHg systolic.

### Embodiment 14

A method for
(i) the treatment or prevention of at least one complication associated with apnoea in a subject; and/or
(ii) increasing apnoea tolerance in a subject,
   wherein the apnoea is during, or expected during, anaesthesia, comprising administering to the subject a composition of the first aspect of the invention.

### Embodiment 15

The method of embodiment 13, wherein the surgery is selected from the group consisting of ophthalmic surgery, neurosurgery, vascular surgery and otorhinolaryngology surgery on the middle ear, amongst others.

### Embodiment 16

The method of embodiment 53, wherein the surgery is vascular surgery that requires relaxed blood vessels, and the surgery involves arterial catheterisation, preferably wherein the surgery involves cardiac angiogram, cardiac stent placement, rotablation, and/or intra-arterial balloon tamponading.

### Embodiment 17

The method of embodiment 13 or embodiments 14, wherein the use is in, or prior to, surgery, and the surgery is bariatric surgery.

### Embodiment 18

The method of any one of embodiments 13 to 17, wherein the at least one complication is selected from the group consisting of oxygen deficiency, such as limited blood flow or oxygen to the heart or brain and cerebral hypoxia; high blood pressure; diabetes; transient ischemic attack (TIA); stroke; heart attack; and death.

### Embodiment 19

The method of any one of embodiments 13 to 18, wherein the composition is administered to the subject before and/or during surgery, preferably before surgery, more preferably before both surgery and anaesthesia.

### Embodiment 20

The method of any one of embodiments 13 to 19, wherein the subject
(i) is obese, preferably having a Body Mass Index (BMI) of greater than 35 kg/m³; and/or
(ii) has low functional residual lung capacity (FRC), preferably an FRC of less than 30 mL/kg when measured in the supine position.

### Embodiment 21

The method of any one of embodiments 13 to 20, wherein, in use, the composition is administered to the subject under normobaric conditions.

### Embodiment 22

The method of any one of embodiments 13 to 21, wherein the use is when the subject is under general anaesthesia, or before general anaesthesia is administered, preferably before general anaesthesia is administered.

### Embodiment 23

The method of any one of embodiments 13 to 22, wherein the composition is administered to the subject as a gas by inhalation.

### Embodiment 24

The method of any one of embodiments 13 to 23, wherein the composition consists of 95% by volume of O₂ and 5% by volume of CO₂.

The invention will now be further illustrated by the following non-limiting examples. The specific examples below are to be construed as merely illustrative, and not limitative of the remainder of the disclosure in any way whatsoever. Without further elaboration, it is believed that one skilled in the art can, based on the description herein, utilise the present invention to its fullest extent. All references and publications cited herein are hereby incorporated by reference in their entirety.

### Example 1

### Study Design and Participants

Thirty participants were scheduled for elective primary bariatric surgery under general anaesthesia. The inclusion criteria include having an age between 18 and 65 years, and a BMI>35 kg/m². Exclusion criteria were severe end-organ damage; chronic obstructive pulmonary disease (COPD) GOLD III and IV or other chronic respiratory disease; known hepatic insufficiency or liver enzymes>50% over the upper reference value of the University Hospital Zurich; renal creatinine clearance<30 mL/min; diagnosed pulmonary hypertension (mean pulmonary arterial pressure≥25 mmHg); severe cardiovascular disease (New York Heart Association, NYHA classification III and IV); history of cerebrovascular disease; drug- or alcohol abuse; and pregnancy. In patients at risk for cardiovascular disease (age>50 years, history of atherosclerosis, or any related diseases) significant vascular stenosis (>50%) of the carotid arteries was excluded by duplex examination.

### Randomization and Blinding

Patients were randomized on the day of their scheduled surgery. The patients were randomly allocated (1:1) to receive the control (95% O₂) followed by 5% CO₂/95% O₂ or vice-versa. The patients were blinded to the intervention, but blinding of the investigators was not feasible.

### Preparation and Induction

Upon arrival in the operating theatre, patients were monitored with peripheral oxygen saturation (SpO₂) monitoring, electrocardiogram (ECG), venous and arterial lines, bispectral index (BIS), and near-infrared spectroscopy (NIRS) for tissue oxygen index (TOI) measurement. The NIRS and BIS sensors were placed on the patients' left and right forehead, respectively. Patients were preoxygenated with 100% O₂ until an expiratory fraction of oxygen of ≥90% was reached, after which rapid sequence induction was performed, using fentanyl 2-3 μg/kg, propofol 2 mg/kg, and rocuronium 0.9 mg/kg intravenously applied. The airway was secured by an endotracheal tube placed with C-MAC (Storz, Tuttlingen, Germany). General anaesthesia was then maintained with propofol 4-10 mg/kg/h, and BIS monitoring was used to ensure a constant depth of anaesthesia throughout the study intervention (BIS target range 30-50).

### Study Intervention

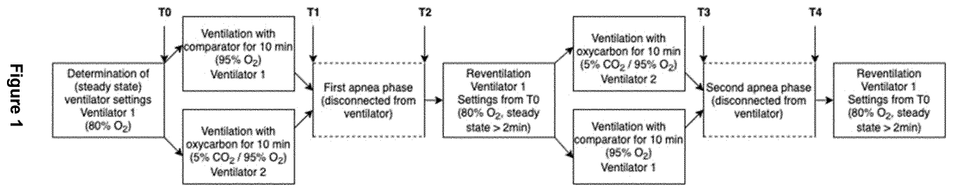

An overview over the study intervention is given in FIG. 1.

### T0

After induction, patients were ventilated (Dräger Primus Infinity Empowered, Dräger, Lübeck, Germany) in a volume-controlled mode with an inspiratory oxygen fraction (FiO₂) of 0.8 (unless a higher FiO₂ was necessary for normoxia), a peak inspiratory pressure of 15-35 mmHg, and respiratory rate of 10-18 breaths per minute (bpm), in order to obtain normocapnic end-tidal CO₂ (EtCO₂) values of 4.5-5.5 kPa over at least 2 minutes. When this steady-state was reached, it was defined as time-point 0 (T0), and baseline values and ventilator settings were recorded.

### T1

Patients were then first connected either to ventilator 1 (“comparator”, with 95% O₂) or to ventilator 2 (5% CO₂/95% O₂) according to the allocated treatment sequence. Ventilator 1 was a Dräger Primus Infinity Empowered, and ventilator 2 a Dräger Evita Infinity V500 (both from Dräger), both ventilators were comparable. Ventilator 1 was connected to O₂ and compressed air wall supply, while ventilator 2 was connected to an 5% CO₂/95% O₂ gas bottle produced by Linde Group (Dublin, Ireland).

Patients were ventilated for at least 10 minutes with the comparator or 5% CO₂/95% O₂ gas. The fraction of expired O₂ (FeO₂) had to reach ≥80% and to be stable for more than one minute. To initiate the apnoea, the endotracheal tube was disconnected from the ventilator, until TOI had dropped 20% from baseline (primary endpoint). For safety reasons, the apnoea phase was terminated early if a TOI below 50% absolute value or a SpO₂ of 80% were reached (safety endpoint). The TOI values were chosen according to the institutional standards applied for carotid endarterectomy procedures.

T1 corresponds to the initiation of the apnoea phase.

### T2

T2 was defined as the time-point at the end of the apnoea phase when ventilation was resumed. Re-ventilation was performed with the ventilator settings used at T0, to allow the patient to return to the steady state with an EtCO₂ of 4.5-5.5 kPa for at least 2 minutes. Then the cross-over ventilation was initiated for 10 min.

### T3 and T4

T3 corresponds to the beginning of the second apnoea phase, and T4 to the end of the second apnoea phase, when the patient was re-ventilated.

At all time-points (T0 to T4), blood gas samples were obtained, and PaO₂ and PaCO₂ were measured using the ABL835 Flex blood gas analyser from Radiometer Medical (Copenhagen, Denmark). Further, SpO₂, TOI, and BIS were recorded.

### End of Study Intervention and Follow-Up

After T4, upon reaching steady-state, the study intervention was completed, and bariatric surgery was started. After surgery patients were transferred to the post-anaesthesia care unit and two hours later to the surgical ward. To exclude study-related adverse (AE) or serious adverse event (SAE), each patient was followed-up after 24 hours. In case of an AE or SAE, the patient was followed-up until the AE or SAE was resolved.

### Outcomes

Primary outcome of this study was the length of the apnoea phase until TOI decreased by 20% from baseline (assessed by NIRS). Differences in PaO₂, PaCO₂, SpO₂, NIRS values (in case the primary endpoint could not be reached), BIS values, heart rate (HR), and mean arterial pressure (MAP) were defined as secondary outcomes.

### Statistical Analyses

For the power calculation, data variability has been estimated using data from Eichhorn et al. J Clin Monit Comput 2015, 29(6):749-757. Assuming a within subject correlation of p=0.3, a standard deviation of 45.04 was calculated (Julious SA Stat Med 2004, 23(12):1921-1986). Using the formula published by Senn (Cross-over trials in clinical research, 2nd edn. Chichester, Eng.; New York: J. Wiley; 2002), a total of 28 patients are needed to show a difference of 30 seconds between the two treatments (assuming a dropout rate of 5%, the target sample size of 30 was determined).

Continuous data were summarised as median and interquartile range (IQR), and categorical data were summarized as numbers (n) and proportions of the total (%). Linear mixed-effects models were used to estimate the average effect of 5% CO₂/95% O₂ gas adjusted for potential period effects, while including the patient ID as a random effect. Other models were additionally adjusted for baseline measurements or the presence of obstructive sleep apnoea. As baseline-correction did not impact on the study results, all data are presented without baseline correction in the final analyses.

Linear mixed models with and without adjustment for additional co-variables, model diagnostics, as well as visualisation methods, adhere to the recommendations published by Senn (supra). Pearson correlation was used to quantify the linear association between study outcomes at T2 and T4. As presumed in the trial protocol, missing data did not exceed 5% in any of the primary or secondary outcomes. While no imputation methods were considered, the models included all patients even if missing values were present in one of the two intervention periods. All analyses were performed in the R programming language (R Core Team, 2017) (R version 3.5.2 (2018-12-20)). Linear mixed-effects models were fit using the Ime4 package (Bates D et al. J Stat Softw 2015, 67(1):1-48) with p-values computed using the ImerTest package (Kuznetsova A et al. J Stat Softw 2017, 82(13):1-26).

### Results

Data Collection and Presentation

Two patients, one in the comparator-5% CO₂/95% O₂ gas group, and one in the 5% CO₂/95% O₂ gas-comparator group could not undergo the second intervention due to bronchospasm during the first re-ventilation phase. Two patients started to breathe spontaneously during the apnoea phase, and in one patient, a blood sample was drawn not at the end of the apnoea phase, but when re-ventilation had already started.

### Patient Characteristics and Baseline Parameters

Patient characteristics including age, sex, BMI, as well as comorbidities (arterial hypertension, diabetes, NYHA class) and laboratory parameters were balanced between the two randomisation sequences, except for the incidence of sleep apnoea, which was higher in the comparator-5% CO₂/95% O₂ group than in the 5% CO₂/95% O₂-comparator group. All patient characteristics are presented in Table 1.

TABLE 1
patient characteristics

| | | control-5% CO₂/95% O₂ | 5% CO₂/95% O₂-control |
|---|---|---|---|
| n | | 17 | 13 |
| Age | | 41 [31, 51] | 43 [34, 52] |
| Sex | female | 11 (65) | 10 (77) |
| BMI | | 45.0 [40.6, 51.4] | 42.6 [40.8, 47.1] |
| Arterial | | 9 (53) | 6 (46) |
| hypertension | | | |
| Diabetes | | 2 (12) | 2(15) |
| Sleep apnoea | | 9 (53) | 2(15) |
| NYHA class | 0 | 14 (82) | 11 (84) |
| | 1 | 0 (0) | 1 (8) |
| | 2 | 3 (18) | 1 (8) |
| Platelets | | | |
| Haemoglobin | | 143 [132, 150] | 138 [125, 145] |
| Haematocrit | | 0.43 [0.42, 0.45] | 0.43 [0.39, 0.44] |
| Thrombocyte | | 243 [227, 327] | 278 [245, 328] |
| INR | | 1.00 [1.00, 1.05] | 1.10 [1.00, 1.10] |
| Sodium | | 139 [138, 141] | 140 [138, 141] |
| Potassium | | 4.1 [3.9, 4.3] | 3.9 [3.7, 4.2] |
| Creatinine | | 70 [59, 87] | 68 [64, 84] |

(Data reported as abso ute number (percentage) and median [Q1, Q3] for the two intervention-sequences control-5% CO₂/95%O₂ and 5% CO₂/95% O₂-control.)

At baseline (T0), TOI, BIS values, MAP, heart rate, SpO₂, EtCO₂, respiratory rate, airway pressures, PaCO₂, and PaO₂ were comparable between the two randomisation sequences, while the comparator-first group had a higher FiO₂ and minute ventilation than the 5% CO₂/95% O₂-comparator group. Detailed information regarding the neurological activity, vital parameters, and ventilator settings at T0 are provided in Table 2.

TABLE 2
Baseline values and settings at T0

| | control-5% CO₂/95% O₂ | 5% CO₂/95% O₂-control |
|---|---|---|
| n | 17 | 13 |
| TOI | 82 [78, 84] | 81 [79, 84] |
| BIS | 38 [29, 42] | 35 [27, 40] |
| MAP | 69 [63, 78] | 71 [67, 74] |
| Heart Rate | 71 [66, 84] | 80 [74, 93] |
| SpO₂ | 98 [97, 98] | 98 [96, 98] |
| FiO₂ | 84 [80, 92] | 80 [77, 80] |
| ETCO₂ | 4.8 [4.6, 5.0] | 4.9 [4.7, 5.1] |
| Respiratory Rate | 14 [13, 16] | 13 [12, 14] |
| MV | 7.1 [6.4, 8.0] | 6.0 [5.9, 6.4] |
| PEEP | 8 [7, 8] | 8 [7, 8] |
| PIP | 23 [21,27] | 22 [20, 23] |
| PCO₂ | 5.15 [5.06, 5.72] | 5.49 [5.04, 5.65] |
| PaO₂ | 24.9 [18.2, 36.7] | 25.7 [24.1,27.6] |

(Data reported as median [Q1, Q3]. Data described in table 2 were recorded after reaching a steady-state at time-point T0 when ventilating patients with a fraction of inspired oxygen (FiO₂) of 80%, before starting the wash-in of the first study gas.)

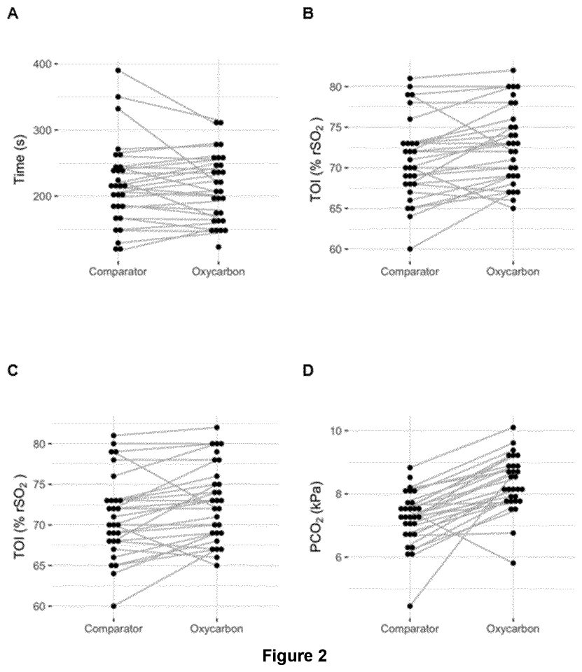

The time until SpO₂ dropped to 80% was similar after ventilation with 5% CO₂/95% O₂ (vs. comparator), with a mean difference of −6 s (95%Cl: from −19 to 7; p=0.37; FIG. 2A).

At the end of the apnoea phase (T2 and T4, respectively) cerebral TOI was significantly higher after 5% CO₂/95% O₂, with a mean difference of 1.5% (95%Cl: from 0.3 to 2.6; p=0.02; FIG. 2B). Similarly, 5% CO₂/95% O₂ was also associated with a higher PaO₂ (at T2 and T4, respectively, mean difference 0.6 kPa; 95%Cl: from 0.1 to 1.1; p=0.02; FIG. 2C) and higher PaCO₂ at the end of the apnoea phase (mean difference 1.06 kPa; 95%Cl: from 0.76 to 1.36; p<0.001; FIG. 2D).

BIS, MAP, and HR were comparable after ventilation with 5% CO₂/95% O₂ (vs. comparator), with mean differences of +1% BIS (95%Cl: from −3 to 5; p=0.7), −3 mmHg MAP (95%Cl: from −9 to 3; p=0.3), +1/min HR (95%Cl: from −1 to 4; p=0.3) respectively.

Additionally, the lowest TOI value for both interventions was defined in each subject, and the time to reach it after apnoea initiation was determined. On average, time to lowest TOI was 9 seconds longer under 5% CO₂/95% O₂ than under the comparator (95%Cl: from −2 to 20).

### Carry-Over Effect

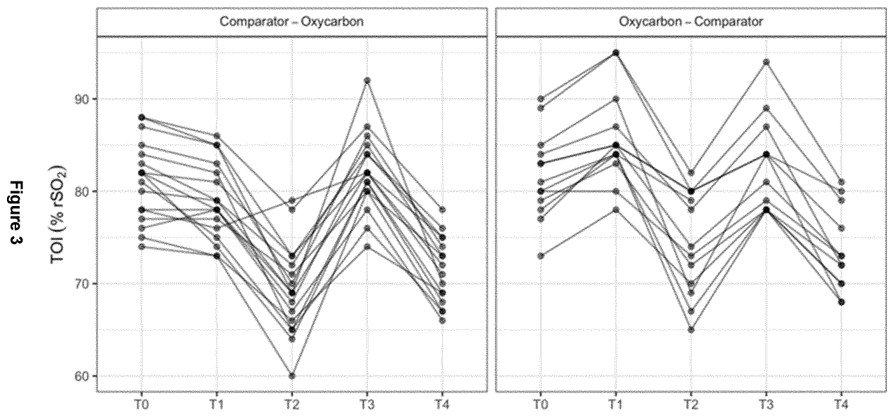

A further unexpected advantage was seen in a carry-over effect that was observed for patients who received 5% CO₂/95% O₂ as the first intervention. In these patients, the TOI values increased from T0 to T1, after ventilation with 5% CO₂/95% O₂ for the first intervention, but remained at the level they reached at T1 when ventilated with the comparator for the second intervention, before the second apnoea phase (T3). It is surprising that the effect of 5% CO₂/95% O₂ on cerebral TOI was still present even after the wash-out period and the wash-in of the comparator. On the contrary, for patients who were allocated to the comparator-5% CO₂/95% O₂ group, TOI values were similar at T0 and T1, and higher at T3, after ventilation with 5% CO₂/95% O₂ before the second apnoea phase. The carry-over effect is depicted in FIG. 3. This supports the administration of the composition of the invention before surgery and/or anaesthesia.

The following are non-limiting numbered embodiments of the invention.

Numbered embodiment 1. A composition comprising oxygen (O₂) and carbon dioxide (CO₂), wherein
(i) O₂ is present in an amount of from about 91% to about 99% by volume, preferably from about 93% to about 97% by volume; and
(ii) CO₂ is present in an amount of from about 1% to about 9% by volume, preferably from about 3% to about 7% by volume,
   based upon the total volume of the composition.

Numbered embodiment 2. The composition of numbered embodiment 1, wherein the composition comprises carrier gas in an amount of from greater than 0% to about 8% by volume based upon the total volume of the composition, preferably wherein the carrier gas comprises nitrogen (N₂), helium (He), xenon (Xe), argon (Ar), krypton (Kr) or a mixture thereof.

Numbered embodiment 3. The composition of numbered embodiment 1, wherein the composition does not consist of 95% by volume of O₂ and 5% by volume of CO₂ based upon the total volume of the composition.

Numbered embodiment 4. A composition of any one of numbered embodiments 1 to 3 for use as a medicament.

Numbered embodiment 5. A composition of any one of numbered embodiments 1 to 3 for use
(i) in the treatment or prevention of at least one complication associated with surgery on a subject; and/or
(ii) in, or prior to, surgery on a subject,
   wherein it is desirable that the subject has low blood pressure during the surgery and/or the surgery is expected to result in high blood loss, preferably wherein the blood pressure is less than 90 mmHg systolic.

Numbered embodiment 6. A composition of any one of numbered embodiments 1 to 3 for use in
(i) the treatment or prevention of at least one complication associated with apnoea in a subject; and/or
(ii) increasing apnoea tolerance in a subject,
   wherein the apnoea is during, or expected during, anaesthesia.

Numbered embodiment 7. The composition for use of numbered embodiment 5, wherein the surgery is selected from the group consisting of ophthalmic surgery, neurosurgery, vascular surgery and otorhinolaryngology surgery on the middle ear, amongst others.

Numbered embodiment 8. The composition for use of numbered embodiment 5 or numbered embodiment 6, wherein the use is in, or prior to, surgery, and the surgery is bariatric surgery.

Numbered embodiment 9. The composition for use of any one of numbered embodiments 5 to 8, wherein at least one complication is selected from the group consisting of oxygen deficiency, such as limited blood flow or oxygen to the heart or brain and cerebral hypoxia; high blood pressure; diabetes; transient ischemic attack (TIA); stroke; heart attack; and death.

Numbered embodiment 10. The composition for use of any one of numbered embodiments 5 to 9, wherein the composition is administered to the subject before and/or during surgery, preferably before surgery, more preferably before both surgery and anaesthesia.

Numbered embodiment 11. The composition for use of any one of numbered embodiments 5 to 10, wherein the subject
(i) is obese, preferably having a Body Mass Index (BMI) of greater than 35 kg/m³; and/or
(ii) has low functional residual lung capacity (FRC), preferably an FRC of less than 30 mL/kg when measured in the supine position.

Numbered embodiment 12. The composition for use of any one of numbered embodiments 5 to 11, wherein, in use, the composition is administered to the subject under normobaric conditions.

Numbered embodiment 13. The composition for use of any one of numbered embodiments 5 to 12, wherein the use is when the subject is under general anaesthesia, or before general anaesthesia is administered, preferably before general anaesthesia is administered.

Numbered embodiment 14. The composition for use of any one of numbered embodiments 5 to 13, wherein the composition is administered to the subject as a gas by inhalation.

Numbered embodiment 15. The composition for use of any one of numbered embodiments 5 to 14, wherein the composition consists of 95% by volume of O₂ and 5% by volume of CO₂.

1. A composition for use
(i) in the treatment or prevention of at least one complication associated with surgery on a subject; and/or
(ii) in, or prior to, surgery on a subject,
wherein it is desirable that the subject has low blood pressure during the surgery; the surgery is expected to result in high blood loss; and/or the surgery requires relaxed blood vessels, preferably wherein the blood pressure is less than 90 mmHg systolic;
wherein the composition comprises oxygen (O₂) and carbon dioxide (CO₂), wherein
(A) O₂ is present in an amount of from about 91% to about 99% by volume, preferably from about 93% to about 97% by volume; and
(B) CO₂ is present in an amount of from about 1% to about 9% by volume, preferably from about 3% to about 7% by volume,
based upon the total volume of the composition.

2. A composition for use in
(i) the treatment or prevention of at least one complication associated with apnoea in a subject; and/or
(ii) increasing apnoea tolerance in a subject,
wherein the apnoea is during, or expected during, anaesthesia;
wherein the composition comprises O₂ and CO₂, wherein
(A) O₂ is present in an amount of from about 91% to about 99% by volume, preferably from about 93% to about 97% by volume; and
(B) CO₂ is present in an amount of from about 1% to about 9% by volume, preferably from about 3% to about 7% by volume,
based upon the total volume of the composition.

3. The composition for use in claim 1, wherein the surgery is selected from the group consisting of ophthalmic surgery, neurosurgery, vascular surgery and otorhinolaryngology surgery on the middle ear, amongst others.

4. The composition for use as claimed in claim 3, wherein the surgery is vascular surgery, the surgery requires relaxed blood vessels, and the surgery involves arterial catheterisation, preferably wherein the surgery involves cardiac angiogram, cardiac stent placement, rotablation, and/or intra-arterial balloon tamponading.

5. The composition for use as claimed in claim 1, wherein the use is in, or prior to, surgery, and the surgery is bariatric surgery.

6. The composition for use as claimed in claim 1, wherein the at least one complication is selected from the group consisting of oxygen deficiency, such as limited blood flow or oxygen to the heart or brain and cerebral hypoxia; high blood pressure; diabetes; transient ischemic attack (TIA); stroke; heart attack; and death.

7. The composition for use as claimed in claim 1, wherein the composition is administered to the subject before and/or during surgery, preferably before surgery, more preferably before both surgery and anaesthesia.

8. The composition for use as claimed in claim 1, wherein the subject
(i) is obese, preferably having a Body Mass Index (BMI) of greater than 35 kg/m³; and/or
(ii) has low functional residual lung capacity (FRC), preferably an FRC of less than 30 mL/kg when measured in the supine position.

9. The composition for use as claimed in claim 1, wherein, in use, the composition is administered to the subject under normobaric conditions.

10. The composition for use as claimed in claim 1, wherein the use is when the subject is under general anaesthesia, or before general anaesthesia is administered, preferably before general anaesthesia is administered.

11. The composition for use as claimed in claim 1, wherein the composition is administered to the subject as a gas by inhalation.

12. The composition for use as claimed in claim 1, wherein the composition consists of 95% by volume of O₂ and 5% by volume of CO₂.

13. The composition for use as claimed in claim 1, wherein the composition comprises carrier gas in an amount of from greater than 0% to about 8% by volume based upon the total volume of the composition, preferably wherein the carrier gas comprises nitrogen (N₂), helium (He), xenon (Xe), argon (Ar), krypton (Kr) or a mixture thereof.

14. The composition for use as claimed in claim 1, wherein the composition does not consist of 95% by volume of O₂ and 5% by volume of CO₂ based upon the total volume of the composition.

15. A composition comprising O₂ and CO₂, wherein
(i) O₂ is present in an amount of from about 91% to about 99% by volume, preferably from about 93% to about 97% by volume; and
(ii) CO₂ is present in an amount of from about 1% to about 9% by volume, preferably from about 3% to about 7% by volume,
based upon the total volume of the composition.

16. The composition as claimed in claim 15, wherein the composition comprises carrier gas in an amount of from greater than 0% to about 8% by volume based upon the total volume of the composition, preferably wherein the carrier gas comprises N₂, He, Xe, Ar, Kr or a mixture thereof.

17. The composition as claimed in claim 15, wherein the composition does not consist of 95% by volume of O₂ and 5% by volume of CO₂ based upon the total volume of the composition.

18. A composition as claimed in claim 15 for use as a medicament.
